(19) United States
(12) Patent Application Publication
(10) Pub. No.: US 2019/0194122 A1
(43) Pub. Date: Jun. 27, 2019

(54) Processes for the Preparation of Dasotraline and Intermediates Thereof

(71) Applicant: Apotex Inc., Toronto (CA)

(72) Inventors: Prabhudas Bodhuri, Torrance, CA (US); Eduardo Gustavo Cammisa, Markham (CA); Melanie R.A. Green, Milton (CA); Gamini Weeratunga, Ancaster (CA); Boris Gorin, Oakville (CA)

(21) Appl. No.: 16/223,326

(22) Filed: Dec. 18, 2018

Related U.S. Application Data
(60) Provisional application No. 62/610,608, filed on Dec. 27, 2017.

(51) Int. Cl. C07C 209/70; C07C 209/62; C07C 209/28; C07B 53/00

(52) U.S. Cl. CPC C07C 209/70; C07C 209/62; C07C 211/42; C07B 53/00; C07C 209/28

(57) ABSTRACT

The present invention provides processes for the preparation of Dasotraline (1), as well as intermediates useful in the preparation thereof. In particular, processes are provided for the production of the compound of Formula (2), or a salt thereof, and its deprotection to afford Dasotraline (1).

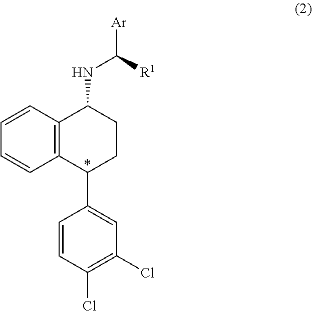

### CROSS-REFERENCE TO RELATED APPLICATION

This application claims the benefit of U.S. Provisional Patent Application No. 62/610,608, filed Dec. 27, 2017, the disclosure of which is hereby incorporated in its entirety by reference.

### TECHNICAL FIELD

The present invention relates to processes for the preparation of Dasotraline (1) and intermediates used in the preparation thereof.

### BACKGROUND

Dasotraline (1), or (1R,4S)-4-(3,4-dichlorophenyl)-1,2,3,4-tetrahydro-1-naphthalenamine, acts as a dual dopamine and norepinephrine reuptake inhibitor (DNRI), and is undergoing evaluation as a treatment for attention deficit hyperactivity disorder (ADHD) as its hydrochloride salt.

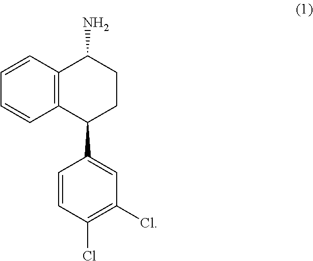 [SMILES: N[C@@H]1CC[C@@H](c2ccc(Cl)c(Cl)c2)c2ccccc21]

Scheme 1 depicts a known method of preparing the hydrochloride salt of Dasotraline (1) that is described in WO 2004/024669 A1 and Han, Z. et al. Organic Process Research & Development 2007, 11, 726. In this method, chiral (S)-tetralone (E) was prepared from racemic tetralone (A) by chromatographic separation of the sulfinyl imine diastereomers (C), followed by hydrolysis. The resulting (S)-tetralone was converted to a diastereomeric mixture of N-formyl amines (F), which were separated by chromatography to give the desired (1R,4S)-trans-diastereomer (G), which afforded Dasotraline (1) as the hydrochloride salt following acidic hydrolysis. However, there are limitations associated with this process that impede its use on an industrial scale. These limitations include a low overall yield of <2% starting from racemic tetralone (A), which is due, in part, to the loss of at least 50% of the undesired stereoisomer in each of the two steps that generate chirality, as well as the hydrolytic sensitivity of intermediate sulfinyl imine (C). Also impeding the use of this process on an industrial scale is the necessity for two chromatographic separations to isolate diastereomeric intermediates (D) and (G) from mixtures (C) and (F), respectively, particularly owing to the reported sensitivity of sulfinyl imine (C) to hydrolysis.

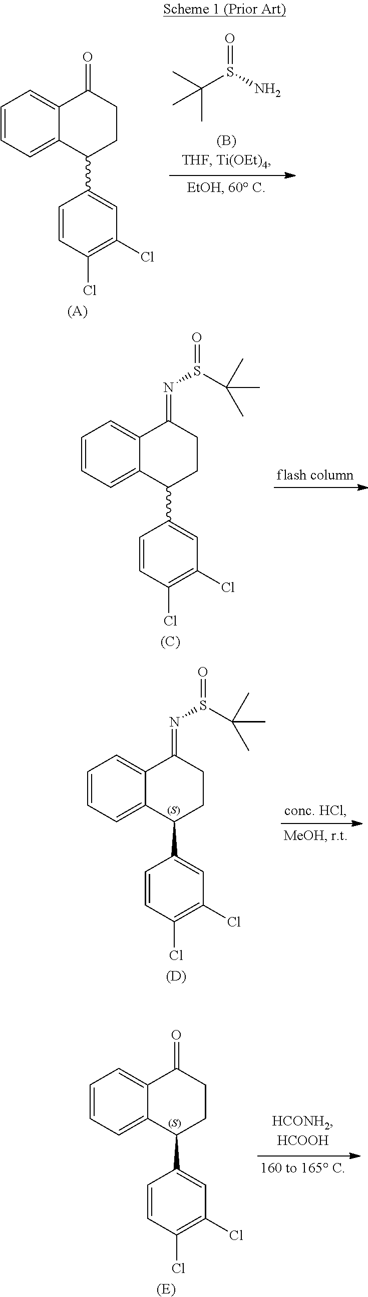

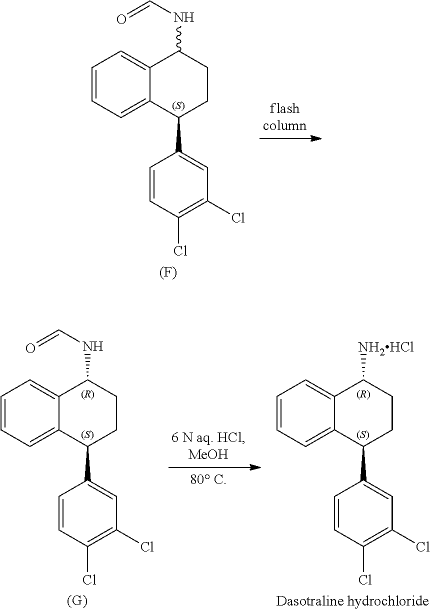

In Han, Z. et al. Organic Process Research & Development 2007, 11, 726, improvements to the route disclosed in WO 2004/024669 Al are presented for the multi-kilogram production of Dasotraline hydrochloride. In this large-scale synthesis, chiral sulfinyl imine (D) is prepared from (S)-tetralone (E) and undergoes direct stereoselective reduction to the corresponding sulfinamide followed by hydrolysis to afford Dasotraline (1), without isolation of any sensitive intermediates. However, this approach suffers from the requirement for stoichiometric amounts of costly reagents such as sulfinamide (B) and 9-borabicyclo[3.3.1]nonane (9-BBN), which are necessary to achieve the highest levels of stereoselectivity.

Further syntheses of Dasotraline (1) are disclosed in Thalen, L. K. et al. Chemistry A European Journal 2009, 15, 3403 and WO 2007/115185 A2. Utilizing different substrates as starting materials, both of these approaches involve asymmetric catalytic hydrogenation of a precursor bearing one fixed chiral centre. A key requirement of this approach is the use of costly homogeneous catalyst systems such as the ‘Crabtree catalyst’, [(C₈H₁₂)Ir{P(C₆H₁₁)₃}(C₅H₅N)]PF₆, or Rh-Norphos, which can be difficult to remove due to their solubility in the reaction conditions. This is of particular relevance in the commercial production of Dasotraline (1), as residual transition metals must be controlled to very low levels in pharmaceutical products.

WO 2008/065177 A1 discloses processes for the preparation of desmethylsertraline, which corresponds with the (1S,4S)-cis-isomer of Dasotraline. Scheme 2 depicts one approach from this disclosure that is used to produce desmethylsertraline involving reductive amination of racemic tetralone (A) with p-methoxybenzylamine, to selectively afford an equimolar mixture of the (1S,4S)- and (1R,4R)-cis-isomers of benzylated amine (I), followed by debenzylation and resolution of the resulting mixture of cis-amines using (+)-tartaric acid to yield desmethylsertraline as the (+)-tartrate salt. This approach to an isomer of Dasotraline suffers from many of the same impediments associated with the known methods of producing Dasotraline, including a low overall yield of <3% starting from racemic tetralone (A).

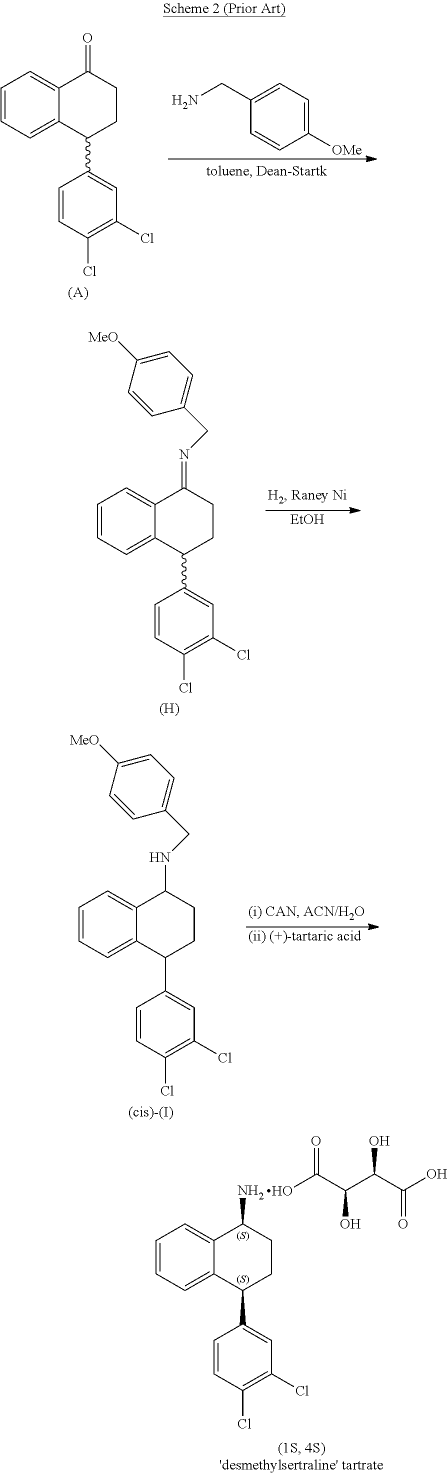

Accordingly, there remains a need in the art for improved processes for the preparation of Dasotraline (1), and the intermediates used in such preparations, that are more amenable to scale-up and use on a commercial scale.

### SUMMARY

The present invention provides improved processes for the preparation of Dasotraline (1), as depicted in Scheme 3. As well, the present invention provides new intermediates of Formulas (2) and (3) that can be used in the preparation of Dasotraline (1), and processes for their preparation.

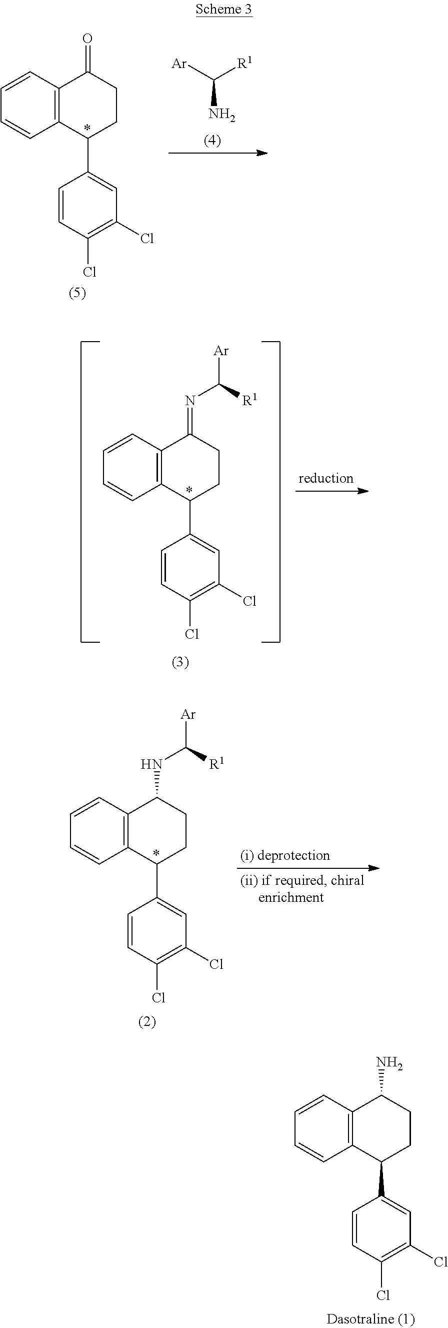

wherein
the carbon atom marked with “*” is racemic, substantially racemic, or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and C₁-C₆ substituted alkyl.

As shown in Scheme 3, Dasotraline (1) may be prepared according to the process of the present invention by a reductive amination between the tetralone of Formula (5) and the amine of Formula (4) to provide a compound of Formula (2). Deprotection of the arylalkyl group of the compound of Formula (2) affords Dasotraline (1), which may be subjected to chiral enrichment procedures, if necessary. In the process of the invention, reduction of the novel imine intermediate of Formula (3) proceeds in favor of the (1R)-isomer to selectively provide the compound of Formula (2).

In one embodiment, the process of the present invention provides Dasotraline (1) having high chiral purity (greater than 95, and preferably greater than 99%) in three steps from a chirally pure tetralone of Formula (5).

In an alternative embodiment, the tetralone of Formula (5) having lower chiral purity in favor of the (S)-isomer, including substantially racemic material, is used as starting material and the selective reductive amination is accompanied by chiral enrichment procedures at one or more stages of the process to provide Dasotraline (1). Preferably, chiral enrichment is performed on the compound of Formula (2). Using this embodiment, the process of the present invention provides Dasotraline (1) selectively with improved yields, and having a chiral purity of >95%, using accessible and affordable reagents without the need for chromatographic purification of sensitive intermediates.

The selectivity of the reduction of the compound of Formula (3) in favour of the (1R)-isomer is particularly surprising given the results reported in WO 2008/065177 A1, wherein reduction of the imine (H) formed between racemic tetralone (A) and p-methoxybenzylamine is presumably driven by the configuration of the C-4 position, and selectively affords an equimolar mixture of the (1S,4S)- and (1R,4R)-isomers over the (1R,4S)-isomer as in the present invention.

Accordingly, in a first aspect of the present invention, there is provided a process for preparing Dasotraline (1), or a salt thereof, comprising deprotection of a compound of Formula (2), or a salt thereof, wherein the carbon atom marked with “*” in the compound of Formula (2) is racemic, substantially racemic, or enantiomerically enriched in the (S)-configuration; Ar is selected from the group consisting of C₆-C₁₂ aryl and C₆-C₁₂ substituted aryl, and R¹ is selected from the group consisting of C₁-C₆ alkyl and C₁-C₆ substituted alkyl.

In a preferred embodiment of the first aspect, Ar in the compound of Formula (2) is selected from the group consisting of phenyl, alkoxy-substituted phenyl, alkyl-substituted phenyl, and naphthyl. More preferably, Ar is 4-methoxyphenyl or phenyl, and most preferably, Ar is 4-methoxyphenyl. In a further preferred embodiment of the first aspect, R¹ in the compound of Formula (2) is methyl.

In another preferred embodiment of the first aspect, the deprotection of the compound of Formula (2) is conducted by a process selected from the group consisting of hydrogenolysis, acid-mediated hydrolysis and oxidative cleavage. In a further preferred embodiment, hydrogenolysis comprises treatment of the compound of Formula (2), in the presence of a hydrogen source, with a metal catalyst. Preferably, the metal catalyst in this embodiment is selected from the group consisting of palladium, palladium hydroxide, ruthenium, and Raney™ nickel. In a further preferred embodiment, acid-mediated hydrolysis comprises treatment of the compound of Formula (2) with an acid. Preferably, the acid in this embodiment is selected from the group consisting of trifluoroacetic acid, hydrochloric acid and trifluoromethanesulfonic acid, and most preferably, trifluoroacetic acid. Preferably, the acid-mediated hydrolysis is conducted at a temperature of at least about 60° C. In a further preferred embodiment, oxidative cleavage comprises treatment of the compound of Formula (2) with ceric ammonium nitrate (CAN).

In another preferred embodiment of the first aspect, the carbon atom in the compound of Formula (2) marked with “*” is enantiomerically enriched in the (S)-configuration, and the compound of Formula (2) has a chiral purity in favour of the (1R,4S)-configuration of at least about 90%. In another preferred embodiment of the first aspect, the carbon atom in the compound of Formula (2) marked with “*” is racemic or substantially racemic. In a further preferred embodiment, the chiral purity of Dasotraline (1) is enhanced further by purification. In this further preferred embodiment, the purification of Dasotraline (1) comprises crystallization from a solvent (S3) selected from the group consisting of acetonitrile, methanol, isopropanol, ethyl acetate, toluene and tetrahydrofuran. In a yet further preferred embodiment, the purification of Dasotraline (1) comprises the formation and isolation of a salt formed between Dasotraline (1) and a chiral acid. Preferably, the chiral acid is selected from the group consisting of (R)-(−)-mandelic acid, (1R)-(−)-10-camphorsulfonic acid and (+)-O,O′-di-p-toluoyl-D-tartaric acid. In a further preferred embodiment, the chiral purity of Dasotraline (1) following purification is at least 95%.

In a second aspect of the present invention, there is provided a process for preparing the compound of Formula (2) or a salt thereof, by reductive amination comprising reaction, in the presence of a solvent (S1), of a compound of Formula (5) with an amine of Formula (4) to afford a compound of Formula (3), followed by reduction, in the presence of a solvent (S2), with a reductant, to afford the compound of Formula (2), or a salt thereof, wherein the carbon atom in the compounds of Formulas (2), (3) and (5) marked with “*” is racemic, substantially racemic or enantiomerically enriched in the (S)-configuration, Ar is selected from the group consisting of C₆-C₁₂ aryl and C₆-C₁₂ substituted aryl, and R¹ is selected from the group consisting of C₁-C₆ alkyl and C₁-C₆ substituted alkyl.

In a preferred embodiment of the second aspect, Ar in the compounds of Formulas (2) and (3) is selected from the group consisting of phenyl, alkoxy substituted phenyl, alkyl substituted phenyl, and naphthyl. More preferably, Ar is 4-methoxyphenyl or phenyl, and most preferably, Ar is 4-methoxyphenyl. In a further preferred embodiment of the second aspect, R¹ in the compounds of Formulas (2) and (3) is methyl.

In another preferred embodiment of the second aspect, the reductant is selected from the group consisting of metal borohydrides, aluminum hydrides, organosilanes in combination with a catalyst, and a hydrogen source in combination with a transition metal catalyst capable of catalyzing hydrogenation reactions. Preferably, the reductant is selected from the group consisting of sodium borohydride, sodium cyanoborohydride and sodium triacetoxyborohydride. In another preferred embodiment of the second aspect, solvent (S1) is toluene. In another preferred embodiment of the second aspect, solvent (S2) is a mixture of toluene and methanol.

In another preferred embodiment of the second aspect, the carbon atom in the compounds of Formulas (2), (3) and (5) marked with “*” is enantiomerically enriched in the (S)-configuration, and the compound of Formula (5) has a chiral purity in favor of the (4S)-configuration of at least about 90%. In another preferred embodiment of the second aspect, the carbon atom marked with “*” is racemic or substantially racemic. In a further preferred embodiment, the chiral purity of the compound of Formula (2) is in favour of the (1R,4S)-configuration, and is enhanced further by purification. In another preferred embodiment, the purification comprises crystallization of an acid salt of the compound of Formula (2) enriched in the (1R,4R)-isomer, and recovery of the mother liquor of the crystallization enriched in the (1R,4S)-isomer of the salt. Preferably, the acid salt is a p-toluenesulfonic acid salt. A further preferred method of purification comprises formation and isolation of a salt formed between the compound of Formula (2) and a chiral acid. Preferably, the chiral acid is selected from the group consisting of (R)-(−)-mandelic acid, (1R)-(−)-10-camphorsulfonic acid and (+)-O,O′-di-p-toluoyl-D-tartaric acid. In a further preferred embodiment, the chiral purity of the compound of Formula (2) is in favour of the (1R,4S)-configuration following purification, and is at least 95%.

Preferably, when practicing the process of the present invention according to the first aspect, the compound of Formula (2) is prepared according to the process of the second aspect of the invention.

In a third aspect of the present invention, there is provided a compound of Formula (2), or a salt thereof, wherein the carbon atom marked with “*” is racemic, substantially racemic, or enantiomerically enriched in the (S)-configuration, Ar is selected from the group consisting of C₆-C₁₂ aryl and C₆-C₁₂ substituted aryl, and R¹ is selected from the group consisting of C₁-C₆ alkyl and substituted C₁-C₆ alkyl.

In a preferred embodiment of the third aspect, Ar is selected from the group consisting of phenyl, alkoxy substituted phenyl, alkyl substituted phenyl, and naphthyl. More preferably, Ar is 4-methoxyphenyl or phenyl, and most preferably, Ar is 4-methoxyphenyl. In a further preferred embodiment of the third aspect, R¹ is methyl.

In another preferred embodiment of the third aspect, the carbon atom marked with “*” is enantiomerically enriched in the (S)-configuration and the chiral purity of the compound in favour of the (1R,4S)-configuration is at least 90%.

In another preferred embodiment of the third aspect, the carbon atom marked with “*” is racemic or substantially racemic.

In another preferred embodiment of the third aspect, the compound of Formula (2) is a p-toluenesulfonic acid salt.

In a fourth aspect of the present invention, there is provided a compound of Formula (3), or a salt thereof, wherein the carbon atom marked with “*” is racemic, substantially racemic, or enantiomerically enriched in the (S)-configuration, Ar is selected from the group consisting of C₆-C₁₂ aryl and C₆-C₁₂ substituted aryl, and R¹ is selected from the group consisting of C₁-C₆ alkyl and C₁-C₆ substituted alkyl.

In a preferred embodiment of the fourth aspect, Ar is selected from the group consisting of phenyl, alkoxy substituted phenyl, alkyl substituted phenyl, and naphthyl. More preferably, Ar is 4-methoxyphenyl or phenyl, and most preferably, Ar is 4-methoxyphenyl. In a further preferred embodiment of the fourth aspect, R¹ is methyl.

In another preferred embodiment of the fourth aspect, the carbon atom marked with “*” is enantiomerically enriched in the (S)-configuration and the chiral purity of the compound in favour of the (1R,4S)-configuration is at least 90%.

In another preferred embodiment of the fourth aspect, the carbon atom marked with “*” is racemic or substantially racemic.

Other aspects and features of the present invention will become apparent to those ordinarily skilled in the art upon review of the following description of specific embodiments of the invention.

### DETAILED DESCRIPTION

The present invention addresses problems associated with the known processes for the preparation of Dasotraline, wherein the inventors have unexpectedly discovered that selective formation of the (1R) chiral centre of the molecule can be achieved by the use of a chiral amine of Formula (4) in the reductive amination of the tetralone precursor of Formula (5). This approach is surprisingly capable of providing the required stereochemical configuration at the C-1 position regardless of the configuration at the C-4 position, despite the suggestion in WO 2008/065177 A1 that reductive amination of the racemic tetralone is controlled by the configuration at C-4, and preferentially yields the undesired (1R,4R)- and (1S,4S)-cis-isomers over the required (1R,4S)-trans-isomer as in the present invention. In preferred embodiments of the present invention wherein the process is applied to the (S)-isomer of the tetralone of Formula (5), a high yielding and short sequence is provided for the preparation of Dasotraline (1) having high chiral purity (greater than 95%, and preferably greater than 99%, of the desired (1R,4S)-isomer). Alternatively, the process of the present invention can be applied to racemic tetralone of Formula (5) affording Dasotraline (1) following resolution of the intermediate compounds of Formulas (2) or (3), and/or Dasotraline (1).

Through the use of the process of the present invention, it is possible to avoid relatively costly reagents that are employed in the known processes for the preparation of Dasotraline (1) and intermediates thereof, such as (R)-tert-butylsulfinamide, catalysts such as the ‘Crabtree catalyst’ ([(C₈H₁₂)Ir{P(C₆H₁₁)₃}(C₅H₅N)]PF₆ ), and 9-BBN. Furthermore, the homogeneous catalyst systems, and the attendant concerns associated with transition metal residues persisting into the final drug substance, are avoided.

As used herein, the term “alkyl”, alone or as part of another substituent, means, unless otherwise stated, a straight or branched chain, saturated hydrocarbon radical having the number of carbon atoms designated (e.g., C₁-C₆ means one to six carbon atoms). When there is no indication of the number of carbon atoms in the alkyl, it is meant, unless otherwise indicated by context, that there are from 1 to 6 carbons. Preferred examples of saturated hydrocarbon groups include methyl, ethyl, n-propyl, iso-propyl, n-butyl, t-butyl, iso-butyl and sec-butyl.

As used herein, the term “aryl”, alone or as part of another substituent, means, unless otherwise stated, a polyunsaturated, aromatic, hydrocarbon radical which can be one or two rings, which are fused together or linked covalently having the number of carbon atoms designated. When there is no indication of the number of carbon atoms in the aryl, it is meant, unless otherwise indicated by context, that there are from 6 to 12 carbons. Preferred examples of aryl groups include phenyl, 1-naphthyl, 2-naphthyl and 4-biphenyl.

As used herein, the term “substituted” refers to the replacement of one or more hydrogen atoms with any one of a variety of substituents. A substituent may be a non-hydrogen atom or multiple atoms, of which at least one is a non-hydrogen atom. A substituted group (e.g., substituted —CH₂CH₃) may be fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). Substituted compounds may comprise substituents selected from the group consisting of: R′, OR′, halogen, CN, NO₂ and CF₃, wherein each R′ is C₁-C₆ alkyl. Preferred examples of substituent groups on substituted aryl and alkyl groups include methyl and methoxy.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

As used herein, “room temperature” refers to a temperature of 20-25° C.

As used herein, the terms “wt %” or “% w/w” refer to a measure of concentration, and is used to express weight solute/total weight as a percentage.

As used herein, the term “% wt/v” refers to a measure of concentration and is used to express weight solute/total volume as a percentage.

As used herein, the term “about” means “close to”, and that variation from the exact value that follows the term is within amounts that a person of skill in the art would understand to be reasonable. For example, when the term “about” is used with respect to temperature, a variation of ±5° C. is generally acceptable when carrying out the processes of the present invention. When used with respect to mole equivalents, a variation of ±0.1 moles is generally acceptable. When used with respect to volumes, a variation of 10% is generally acceptable.

As used herein, the term “substantially racemic” means that there is an enantiomeric excess of less than 10%.

As used herein, the term “chiral purity” refers to the relative amount of the subject isomer with respect to the total amount of isomers of the substance, expressed as an area percentage by HPLC. For example, a compound of Formula (5) having 90% chiral purity in favor of the (4S)-isomer means that, by chiral HPLC, of the total area of peaks corresponding with the compound of Formula (5), the area of the peak corresponding with the (4S)-isomer accounts for 90%, and the area of the peak corresponding with the (4R)-isomer accounts for 10%.

In one embodiment of the present invention, Dasotraline (1) and intermediates useful in the preparation thereof are prepared by the process set out in Scheme 3. Exemplary reagents and conditions for these reactions are disclosed herein.

In the processes described herein, the C₆-C₁₂ aryl group is preferably selected from the group consisting of phenyl, 1-naphthyl, 2-naphthyl and 4-biphenyl, most preferably phenyl. The C₁-C₆ alkyl group is preferably ethyl or methyl, most preferably methyl. Substituted aryl and substituted alkyl groups are preferably substituted with one or more substituents selected from the group consisting of R′, OR′, halogen, CN, NO₂ and CF₃, wherein each R′ is C1-C6 alkyl. Preferably, the substituent is R′ or OR′ and R′ is methyl, and more preferably the substituent is methoxy.

In one embodiment of the present invention, a process is provided for the preparation of the compound of Formula (2):

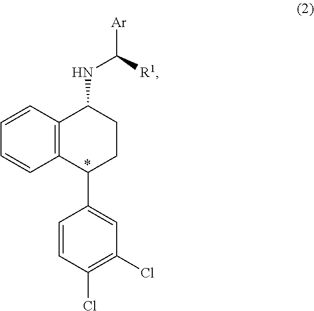

or a salt thereof, by reductive amination comprising reaction, in the presence of a solvent (S1), of a compound of Formula (5):

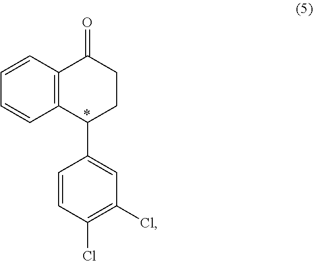 [SMILES: O=C1CCC(c2ccc(Cl)c(Cl)c2)c2ccccc21]

with an amine of Formula (4):

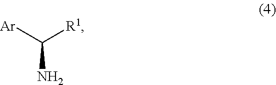

to afford a compound of Formula (3):

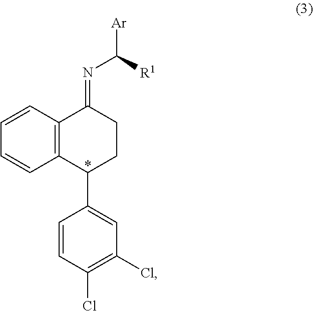

followed by reduction, in the presence of a solvent (S2), with a reductant to afford the compound of Formula (2), or a salt thereof,

wherein
the carbon atom marked with “*” is racemic, substantially racemic or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and C₁-C₆ substituted alkyl.

The reaction of the compound of Formula (5) and the compound of Formula (4) is conducted in the presence of a solvent (S1). Preferably, solvent (S1) is selected to facilitate the reaction as well as to undergo distillation with water that is generated as a by-product of the reaction to promote reaction completion. Alternatively, other mechanisms for the removal of generated water formed during the course of the reaction, such as the use of a suitable drying agent, can be employed. A suitable drying agent is preferably selected from the group consisting of sodium sulfate, magnesium sulfate, and molecular sieves. Solvent (S1) is preferably selected from the group consisting of aromatic hydrocarbons, ethers, chlorinated hydrocarbons and alcohols. More preferably, solvent (S1) is selected from the group consisting of toluene, tetrahydrofuran, dichloromethane, methanol, ethanol and isopropanol. Most preferably, solvent (S1) is toluene.

The reaction of the compound of Formula (5) and the compound of Formula (4) is preferably conducted in the presence of an acid (A1) as a catalyst. Acid (A1) may be any suitable anhydrous acid, and is preferably selected from the group consisting of p-toluenesulfonic acid, hydrochloric acid, methanesulfonic acid, trifluoroacetic acid, acetic acid, formic acid, and acidic ion exchange resins (for example, AMBERLYST®). More preferably, acid (A1) is trifluoroacetic acid.

The reaction of the compound of Formula (5) and the compound of Formula (4) is preferably conducted at a temperature of between room temperature and the boiling point of the reaction mixture, and is preferably between about 20° C. to about 110° C. Preferably, the reaction mixture is refluxed at the boiling point of the reaction mixture when distillation is used to remove water generated in the reaction until water ceases to co-distill.

In the reduction of the compound of Formula (3), the reductant may be selected from the group consisting of metal borohydrides, aluminum hydrides, organosilanes in combination with a catalyst, and a hydrogen source in combination with a transition metal catalyst. Aluminum hydrides are preferably selected from the group consisting of lithium aluminum hydride, diisobutylaluminum hydride and sodium bis(2-methoxyethoxy)aluminum hydride. Organosilanes are preferably selected from the group consisting of polymethylhydrosilane, phenylsilane, dimethylphenylsilane and triethylsilane, and the catalyst used in combination thereof is preferably a Lewis acid catalyst, and most preferably, tris(pentafluorophenyl)borane. When the reductant is a hydrogen source in combination with a transition metal catalyst, the hydrogen source is preferably hydrogen gas, and the transition metal catalyst is preferably selected from the group consisting of palladium, palladium hydroxide, ruthenium, and Raney™ nickel, which may be supported on an inert solid support such as carbon or alumina. Preferably, the hydrogen source is hydrogen gas and the transition metal catalyst is palladium on carbon. Preferably, the reductant is a metal borohydride selected from the group consisting of sodium borohydride, sodium cyanoborohydride (NaBH3CN) and sodium triacetoxyborohydride (NaBH(OAc)₃), and more preferably is sodium borohydride.

The reduction is conducted in the presence of a solvent (S2). Solvent (S2) is preferably selected from the group consisting of alcohols, hydrocarbons, ethers, and mixtures thereof. More preferably, solvent (S2) is selected from the group consisting of methanol, ethanol, toluene, tetrahydrofuran, and mixtures thereof. Most preferably, solvent (S2) is a mixture of methanol and toluene.

With the exception of reductions employing hydrides, the reduction is preferably conducted at a temperature between about room temperature and the boiling point of the reaction mixture, and preferably between about 20° C. to about 60° C. When the reductant is a hydride, the reduction is preferably conducted at a temperature between about −20° C. to about 20° C., and more preferably between about −10° C. to about 0° C.

In one embodiment of the present invention, the reductive amination is conducted in a ‘step-wise’ approach, wherein the compound of Formula (3) is formed in a system comprising solvent (S1), and is isolated prior to the reduction step, conducted in a second system comprising the compound of Formula (3) in solvent (S2).

In a preferred embodiment of the present invention, the reductive amination is conducted in a ‘one-pot’ approach. In this ‘one-pot’ approach, the intermediate imine of Formula (3) is not isolated, but rather, is subjected to reduction directly to afford the compound of Formula (2). In this approach, solvents (S1) and (S2) are chosen to be compatible with each reaction. For example, toluene is preferred during formation of the compound of Formula (3), while methanol or a mixture of methanol and toluene are preferred during reduction. Depending on the choice of reductant, all components of the reaction may be added at once, or the reductant may be added after a period of time has elapsed, to allow formation of imine of Formula (3). In this approach, a suitable temperature may be used that is compatible with both imine formation and reduction.

In a preferred embodiment of the present invention, in the reductive amination of the compound of Formula (5), the carbon marked with “*” is enantiomerically enriched in the (S)-configuration. Preferably the compound of Formula (5) has a chiral purity in favour of the (4S) configuration of at least about 90%, more preferably at least about 95%, and most preferably, at least about 99%, with the same configuration at this carbon at least sustained in the compounds of Formula (2) and (3). In this preferred embodiment, reductive amination of the compound of Formula (5) provides the compound of Formula (2) in favour of the (1R,4S)-trans-configuration.

In another embodiment of the present invention, in the reductive amination of the compound of Formula (5), the carbon marked with “*” is racemic or substantially racemic. In this embodiment, reductive amination of the compound of Formula (5) having approximately equimolar amounts of the (R)- and (S)-configuration at the carbon marked with “*” selectively provides the compound of Formula (2) as a mixture in favour of the (1R,4S)-trans-configuration and the (1R,4R)-cis-configuration, and less of the (1S,4S)-cis- and (1S,4R)-trans-configuration (the configuration of C-1 is selectively (R)).

The chiral purity of the compound of Formula (2), or a salt thereof, whether produced from racemic, substantially racemic or enantiomerically enriched compound of Formula (5), can be further enhanced by purification, preferably by crystallization or by resolution using a chiral acid. Crystallization preferably comprises dissolving or suspending the compound of Formula (2), or a salt thereof, in a suitable solvent (S3) at a suitable temperature, preferably between about 20° C. to about 70° C., followed by cooling, if necessary, and isolation of the resulting solid. Alternatively, in this crystallization process, purified compound of Formula (2), or a salt thereof, may be recovered from the mother liquor, whereas the impurities are enriched in the crystallized solid. Preferably, an acid salt of the compound of Formula (2) with p-toluenesulfonic acid is used for the purification of the compound of Formula (2) by recovery of the mother liquor from a crystallization process.

The suitable solvent (S3) is preferably selected from the group consisting of nitriles, alcohols, esters, aromatic hydrocarbons and ethers; more preferably, solvent (S3) is selected from the group consisting of acetonitrile, methanol, isopropanol, ethyl acetate, toluene and tetrahydrofuran; and most preferably, solvent (S3) is toluene.

Resolution using a chiral acid comprises forming an acid salt of the compound of Formula (2) with a chiral acid in a suitable solvent (S3), as described above, at a suitable temperature, preferably between about 20° C. to about 70° C., followed by cooling, if necessary, and isolation of the resulting solid. Preferably, the chiral acid is selected from the group consisting of the optical isomers of di-p-toluoyl tartaric acid, mandelic acid, 10-camphorsulfonic acid, tartaric acid, dibenzoyl tartaric acid and malic acid. Preferably, the chiral acid is selected from the group consisting of (R)-(−)-mandelic acid, (1R)-(−)-10-camphorsulfonic acid, and (+)-O,O′-di-p-toluoyl-D-tartaric acid; most preferably, the chiral acid is (1R)-(−)-10-camphorsulfonic acid. Following isolation, liberation of the salt according to standard methods, such as dissolution in an aqueous medium, treatment with a base such as sodium hydroxide, sodium bicarbonate or ammonia, and extraction into an organic solvent affords the free compound of Formula (2).

Purification of the compound of Formula (2), or a salt thereof, can be conducted by either or both of these methods. Preferably, following purification, the chiral purity in favour of the (1R,4S)-configuration of the compound of Formula (2), or a salt thereof, is at least 90%, more preferably at least 95%, and most preferably at least 99%.

In another embodiment of the present invention, there is provided a process for preparing Dasotraline (1):

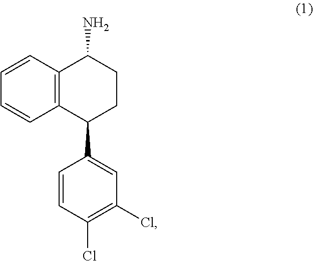 [SMILES: N[C@@H]1CC[C@@H](c2ccc(Cl)c(Cl)c2)c2ccccc21]

or a salt thereof, comprising deprotection of a compound of Formula (2):

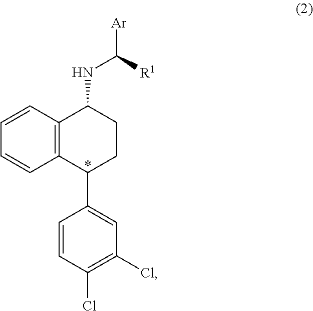

or a salt thereof,

wherein
the carbon atom marked with “*” is racemic, substantially racemic, or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and C₁-C₆ substituted alkyl.

Deprotection of the compound of Formula (2) can be conducted by known processes for the removal of benzylic-type protecting groups from amines, such as those reported in, for example, Greene, T. W.; Wuts, P. G. M. Protective Groups in Organic Synthesis; Fourth edition; Wiley: New York, 2007. Preferably, the deprotection is conducted by a process selected from the group consisting of hydrogenolysis, acid-mediated hydrolysis, and oxidative cleavage (when Ar is alkoxy-substituted).

Deprotection by hydrogenolysis comprises treatment of the compound of Formula (2), in the presence of a hydrogen source, with a transition metal catalyst. The hydrogen source is preferably hydrogen gas, and the transition metal catalyst is preferably selected from the group consisting of palladium, palladium hydroxide, ruthenium, and Raney™ nickel, which may be supported on an inert solid support such as carbon or alumina. Preferably, the transition metal catalyst is palladium on carbon or Raney™ nickel.

Deprotection by hydrogenolysis is conducted in a solvent (S4), which is preferably selected from the group consisting of alcohols and organic acids. More preferably, solvent (S4) is selected from the group consisting of methanol, ethanol, acetic acid, and mixtures thereof.

Deprotection by hydrogenolysis may be conducted at any suitable temperature, preferably between about 20° C. to the boiling point of the reaction mixture, and more preferably at or near the boiling point of the reaction mixture.

Deprotection by acid-mediated hydrolysis comprises treatment of the compound of Formula (2) with an acid, preferably selected from the group consisting of trifluoroacetic acid, hydrochloric acid and trifluoromethanesulfonic acid. Preferably, the acid is trifluoroacetic acid. Preferably, when the acid is liquid, it is used as the solvent for the deprotection. Alternatively, a solvent (S5) is used that is preferably selected from the group consisting of alcohols, water, sulfoxides, formamides, aromatic hydrocarbons, halogenated hydrocarbons, and mixtures thereof.

Deprotection by acid-mediated hydrolysis may be conducted at any suitable temperature, preferably between about 20° C. and the boiling point of the reaction mixture, and more preferably at least about 60° C.

Deprotection by oxidative cleavage comprises treatment of the compound of Formula (2) (when Ar is alkoxy-substituted, preferably methoxy-substituted) with an oxidant selected from the group consisting of ceric ammonium nitrate (CAN), 2,3-dichloro-5,6-dicyanobenzoquinone (DDQ), air, chloranil, and light-induced photoredox in the presence of a catalyst, preferably CAN.

Deprotection by oxidative cleavage using CAN is conducted in a solvent (S6), which is preferably selected from the group consisting of aqueous acetonitrile and acetic acid, and is more preferably aqueous acetonitrile. Deprotection by oxidative cleavage using DDQ is conducted in a solvent (S7), which is preferably selected from the group consisting of toluene, dichloromethane, dioxane, and tetrahydrofuran, and most preferably is dichloromethane.

Deprotection by oxidative cleavage may be conducted at any suitable temperature, preferably between about 20° C. to about 60° C.

In a preferred embodiment of the present invention, in the deprotection of the compound of Formula (2), the carbon marked with “*” is enantiomerically enriched in the (S)-configuration. Preferably the compound of Formula (2) has a chiral purity in favour of the (1R,4S)-configuration of at least about 90%, more preferably at least about 95%, and most preferably at least about 99%.

The chiral purity of Dasotraline (1), or a salt thereof, produced from the compound of Formula (2) can be further enhanced by purification, preferably by direct crystallization or resolution using a chiral acid. Direct crystallization comprises dissolving or suspending Dasotraline (1), or a salt thereof, in a suitable solvent (S3), described above, at a suitable temperature, preferably between about 20° C. to about 70° C., followed by cooling, if necessary, and isolation of the resulting solid. Resolution using a chiral acid comprises forming an acid salt of Dasotraline (1) with a chiral acid in a suitable solvent (S3), as described above, at a suitable temperature, preferably between about 20° C. to about 70° C., followed by cooling, if necessary, and isolation of the resulting solid. Preferably, the chiral acid is selected from the group consisting of the optical isomers of di-p-toluoyl tartaric acid, mandelic acid, 10-camphorsulfonic acid, tartaric acid, dibenzoyl tartaric acid and malic acid. Preferably, the chiral acid is selected from the group consisting of (R)-(−)-mandelic acid, (1R)-(−)-10-camphorsulfonic acid, and (+)-O,O′-di-p-toluoyl-D-tartaric acid, and is most preferably (1R)-(−)-10-camphorsulfonic acid. Following isolation, liberation of the salt according to standard methods, such as dissolution in an aqueous medium, treatment with a base such as sodium hydroxide, sodium bicarbonate or ammonia, and extraction into an organic solvent, affords the free Dasotraline (1).

Purification of Dasotraline (1), or a salt thereof, can be conducted by either or both of these methods. Preferably, following purification, the chiral purity of Dasotraline (1), or a salt thereof, is at least 90%, more preferably at least 95%, and most preferably at least 99%.

In another embodiment of the present invention, there is provided a compound of Formula (2):

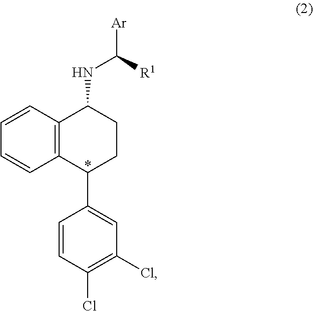

or a salt thereof,

wherein
the carbon atom marked with “*” is racemic, substantially racemic or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and substituted C₁-C₆ alkyl.

In the compound of Formula (2), or a salt thereof, the C₆-C₁₂ aryl group is preferably selected from the group consisting of phenyl, 1-naphthyl, 2-naphthyl and 4-biphenyl, and is most preferably phenyl. The C₁-C₆ alkyl group is preferably ethyl or methyl, and is most preferably methyl. Substituted aryl and substituted alkyl groups are preferably substituted with one or more substituents selected from the group consisting of R′, OR′, halogen, CN, NO₂ and CF₃, wherein each R′ is C₁-C₆ alkyl; more preferably the substituent is methoxy or methyl; and most preferably, the substituent is methoxy.

In the compound of Formula (2), or a salt thereof, Ar is preferably 4-methoxyphenyl or phenyl, and R¹ is methyl. Most preferably, the compound of Formula (2) is the compound of Formula (2-A):

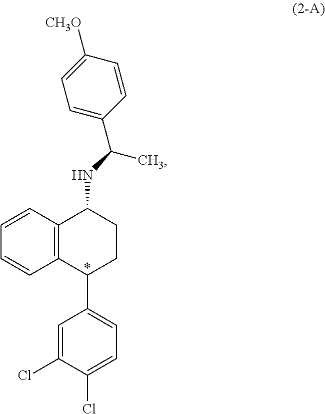

wherein
the carbon atom marked with “*” is racemic, substantially racemic or enantiomerically enriched in the (S)-configuration.

In another embodiment of the present invention, there is provided a compound of Formula (3):

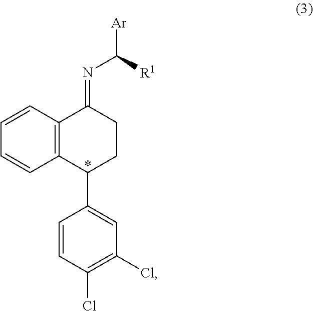

or a salt thereof,

wherein
the carbon atom marked with “*” is substantially racemic or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and C₁-C₆ substituted alkyl.

In the compound of Formula (3), or a salt thereof, the C₆-C₁₂ aryl group is preferably selected from the group consisting of phenyl, 1-naphthyl, 2-naphthyl, and 4-biphenyl, and is most preferably phenyl. The C₁-C₆ alkyl group is preferably ethyl or methyl, and is most preferably methyl. Substituted aryl and substituted alkyl groups are preferably substituted with one or more substituents selected from the group consisting of R′, OR′, halogen, CN, NO₂ and CF₃, wherein each R′ is C₁-C₆ alkyl; more preferably, the substituent is methoxy or methyl; and most preferably, the substituent is methoxy.

In the compound of Formula (3), or a salt thereof, Ar is preferably 4-methoxyphenyl or phenyl, and R¹ is methyl. Most preferably, the compound of Formula (3) is the compound of Formula (3-A):

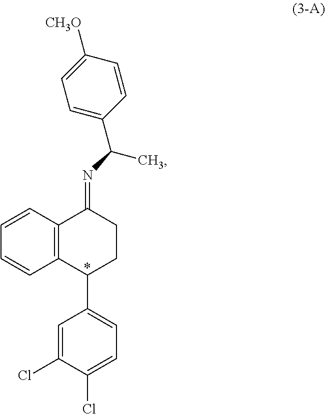

wherein

the carbon atom marked with “*” is racemic, substantially racemic or enantiomerically enriched in the (S)-configuration.

### EXAMPLES

The following examples are illustrative of some of the embodiments of the invention described herein. It will be apparent to the skilled reader that various alterations to the described processes in respect of the reactants, reagents and conditions may be made when using the processes of the present invention without departing from the scope or intent thereof.

### Analysis Method for Determining the Chiral Purity of the Compound of Formula (2):

The method shown in Table 1 was used to determine the chiral purity of samples of Formula (2), including (2-A_{S}) and (2-B_{S}), as provided in the examples that follow.

TABLE 1
HPLC method for the determination of chiral
purity of the compound of Formula (2).

| | |
|---|---|
| Instrument | Agilent 1100 series HPLC |
| Column | Chiralpak AD-H, 4.6 × 250 mm, 5 μm, DAICEL |
| Column Temp. | 25° C. |
| Sample Temp. | 20-25° C. |
| Mobile phase | Mix 900 mL of hexane, 100 mL of ethanol |
| | and 0.7 mL of ethanesulfonic acid (70% |
| | aqueous solution). |
| Mode | Isocratic |
| Flow rate | 1.0 mL/minute |
| Injection volume | 10 μL |
| Detector | 220 nm |
| Run time | 40 minutes |
| Sample prep. | Accurately weigh about 1-2 mg of sample |
| | and dissolve in 1 mL of ethanol, add 2 mL |
| | of mobile phase. |

### Analysis Method for Determination of the Chiral Purity Of Dasotraline (1):

The method shown in Table 2 was used to determine the chiral purity of samples of Dasotraline (1) and salts thereof as provided in the examples that follow.

TABLE 2
HPLC method for the determination of chiral purity
of the compound of Formula (1).

| | |
|---|---|
| Instrument | Agilent 1100 series HPLC |
| Column | Chiralpak AD-H, 4.6 × 250 mm, 5 μm, |
| | DAICEL |
| Column Temp. | 25° C. |
| Sample Temp. | 20-25° C. |
| Mobile phase | Mix 950 mL of hexane, 50 mL of ethanol |
| | and 0.7 mL of ethanesulfonic acid |
| | (70% aqueous solution). |
| Mode | Isocratic |
| Flow rate | 1.0 mL/minute |
| Injection volume | 10 μL |
| Detector | 220 nm |
| Run time | 40 minutes |
| Sample prep. | Accurately weigh about 1-2 mg of sample |
| | and dissolve in 1 mL of ethanol, add |
| | 2 mL of mobile phase. |

### Example 1

### Preparation of (4S)-4-(3,4-dichlorophenyl)-N-[(1R)-1-(4-methoxyphenyl)ethyl]-3,4-dihydronaphthalen-1(2H)-imine (compound of Formula (3-A_{S}))

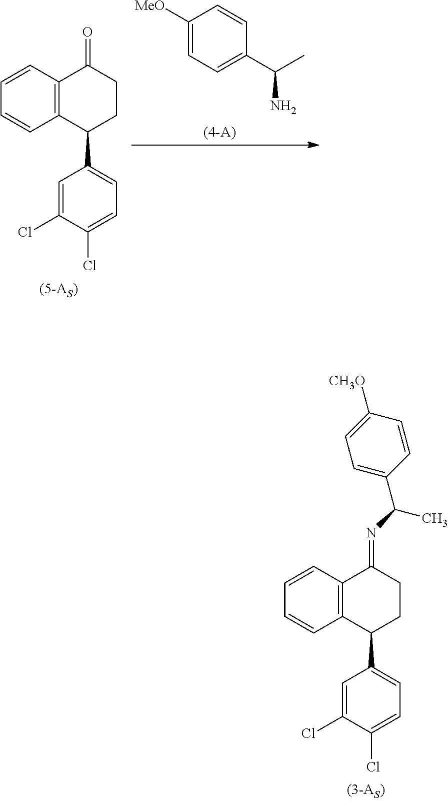

To a solution of the compound of Formula (5-A_{S}) (14.23 g, 48.9 mmol) in toluene (100 mL) was added the compound of Formula (4-A) (8.30 g, 54.9 mmol) and p-toluenesulfonic acid (0.05 g). The resulting solution was refluxed with a Dean-Stark trap for 19 hours, after which time ¹H NMR analysis of a sample of the solution showed 58% conversion. Trifluoroacetic acid (0.2 mL) was added and refluxing was continued for a further 24 hours. The reaction mixture was cooled to room temperature and concentrated to dryness under reduced pressure to afford the compound of Formula (3-A_{S}) as an oil (25.25 g, 82 wt % by assay, 100% yield). The oil was dissolved in toluene to provide a solution (45.2 g solution, 46 wt % (3-A_{S})). Methanol (50 mL) was added to a portion of the toluene solution (8.61 g), containing 3.96 g of the compound of Formula (3-A_{S}). The resulting suspension was heated to reflux until dissolution, cooled to room temperature, and the resulting material was collected by filtration to afford a yellow solid (3.00 g, 76% yield).

¹H-NMR compound of Formula (3-A_{S}): (CDCl₃, 300 MHz) δ: 1.49 (3H, d, J=6.5 Hz), 2.05-2.16 (1H, m), 2.18-2.26 (1H, m), 2.42-2.52 (1H, m), 2.61-2.71 (1H, m), 3.77 (3H, s), 4.08-4.14 (1H, m), 4.76-4.82 (1H, m), 6.84-6.89 (4H, m), 7.16-7.38 (6H, m), 8.39-8.42 (1H, m).

### Example 2

### Preparation of (1R,4S)-4-(3,4-dichlorophenyl)-N-[(1S)-1-(4-methoxyphenyl)ethyl]-1,2,3,4-tetrahydronaphthalen-1-amine (compound of Formula (2-A_{S}))

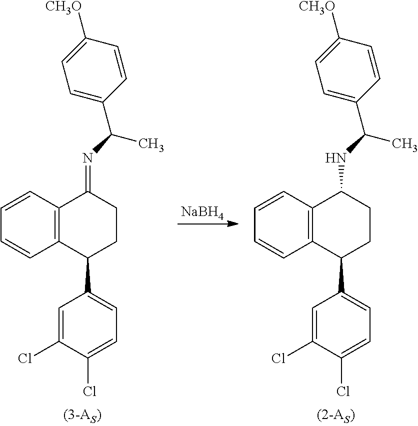

To a toluene solution (36.6 g) of the compound of Formula (3-A_{S}) (46 wt % (3-A_{S}), 40 mmol) prepared in Example 1, was added methanol (90 mL) and the solution cooled to −10° C. Sodium borohydride (1.71 g, 45 mmol) was added over 55 minutes while maintaining the temperature of the reaction mixture below −8° C. After stirring for 2.5 hours at −7° C., a second portion of sodium borohydride (0.82 g, 22 mmol) was added over 45 minutes. After stirring for 16 hours at −4° C., toluene (59 mL) was added, followed by a third portion of sodium borohydride (1.52 g, 40 mmol), which was added over 2.5 hours. The reaction mixture was stirred for 3.5 hours at −5° C. and was subsequently quenched by the addition of concentrated hydrochloric acid (6 N, 12 mL) to adjust the pH to between 1-2. The reaction mixture was allowed to warm to 8° C. and stirred for 20 minutes prior to slow addition of a saturated aqueous solution of sodium bicarbonate (240 mL) to adjust the pH to 8. After stirring for an additional 10 minutes, the phases were separated, and the organic phase was concentrated to dryness to afford the compound of Formula (2-A_{S}) as an oil (18.05 g, 93.6 wt % by assay, 99% yield). Chiral purity (chiral HPLC, area %) in favour of the (1R,4S)-isomer: 93.7% (87.4% enantiomeric excess (ee)).

¹H-NMR compound of Formula (2-A_{S}) (300 MHz, CDCl₃) δ: 1.37 (3H, d, J=6.4 Hz), 1.48-1.56 (1H, m), 1.62-1.68 (1H, m), 1.80-1.88 (1H, m), 2.29-2.38 (1H, m), 3.79-3.85 (1H, m), 3.80 (3H, s), 4.00-4.06 (1H, m), 4.10-4.14 (1H, m), 6.79-6.82 (2H, m), 6.86-6.89 (2H, m), 7.06-7.14 (2H, m), 7.21-7.35 (4H, m), 7.49 (1H, d, J=7.7 Hz).

### Example 3

### Purification of (1R,4S)-4-(3,4-dichlorophenyl)-N-[(1S)-1-(4-methoxyphenyl)ethyl]-1,2,3,4-tetrahydronaphthalen-1-amine (compound of Formula (2-A_{S}))

A sample of the crude compound of Formula (2-A_{S}) prepared in Example 2 (13.70 g, 93.6 wt % by assay) was treated with acetonitrile (15 mL) to afford an oily mixture. Following stirring at room temperature for 1 hour, a suspension had formed, which was stirred for a further period of 2 hours prior to filtration and drying to afford the purified compound of Formula (2-A_{S}) as a white solid (7.78 g, 61% yield). Chromatographic purity (HPLC, area %): 100.0%; chiral purity (chiral HPLC, area %) in favour of the (1R,4S)-isomer: 99.3% (98.6% ee).

### Example 4

### Preparation of (1R,4S)-4-(3,4-dichlorophenyl)-1,2,3,4-tetrahydronaphthalen-1-amine (Dasotraline (1)) Using Trifluoroacetic Acid

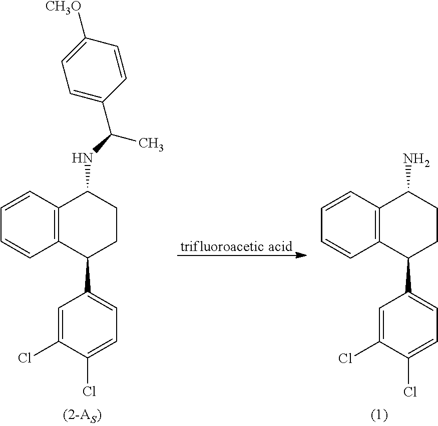

A solution of the compound of Formula (2-A_{S}) (2.07 g, 4.85 mmol) in trifluoroacetic acid (15 mL) was heated for 91 hours at reflux. The reaction mixture was cooled to room temperature, concentrated to dryness, and the residue was diluted with dichloromethane (60 mL). A saturated aqueous solution of sodium bicarbonate (60 mL) was added, and the mixture stirred at room temperature followed by the addition of 10% aqueous potassium carbonate (3 mL) to adjust the pH to 9-10. After phase separation, the organic layer was dried over anhydrous sodium sulfate, filtered, and concentrated to dryness to afford an oil (2.39 g), which was purified by column chromatography (gradient elution: methanol/methylene chloride/ammonium hydroxide from 5/95/1% v/v to 37/63/1% v/v) to afford Dasotraline (1) as an oil (1.30 g, 92% yield). Chromatographic purity (HPLC, area %): 100.0%; chiral purity (chiral HPLC, area %) in favor of the (1R,4S)-isomer: 99.1% (98.2% ee).

¹H-NMR of Dasotraline (1) (300 MHz, CDCl₃) δ: 1.58-1.67 (1H, m), 1.76-1.84 (1H, m), 2.06-2.14 (1H, m), 2.26-2.34 (1H, m), 4.08-4.13 (2H, m), 6.80 (1H, d, J=5.8 Hz), 6.88 (1H, dd, J=6.2, 1.5 Hz), 7.09-7.15 (2H, m), 7.23-7.27 (1H, m), 7.33 (1H, d, J=6.3 Hz), 7.52 (1H, d, J=5.8 Hz).

### Example 5

### Preparation of (1R,4S)-4-(3,4-dichlorophenyl)-1,2,3,4-tetrahydronaphthalen-1-amine (Dasotraline (1)) Using Ceric Ammonium Nitrate (CAN)

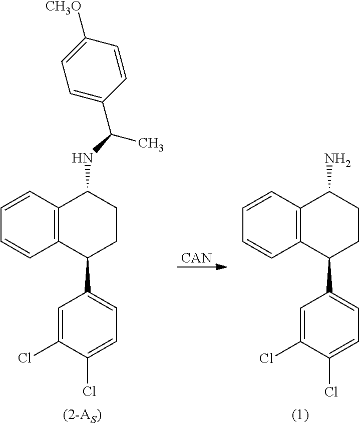

A solution of ceric ammonium nitrate (3.58 g, 6.50 mmol) in water (4 mL) was added dropwise to a solution of the compound of Formula (2-A_{S}) (2.34 g, 5.48 mmol) in acetonitrile (24 mL). The reaction mixture was stirred at room temperature for 4.5 days and monitored by TLC with additional portions of ceric ammonium nitrate added after about 71 hours (3.98 g, 7.30 mmol in 20 mL water) and about 105 hours (4.42 g, 8.06 mmol). Ethyl acetate (250 mL) was added, the phases were separated, and the organic phase was washed with a saturated aqueous solution of sodium bicarbonate (200 mL), a saturated aqueous solution of sodium chloride (150 mL), dried over anhydrous sodium sulfate, and filtered. The filtrate was concentrated to dryness to afford Dasotraline (1) as an oil (1.59 g), which was purified by column chromatography (gradient elution: methanol/methylene chloride/ammonium hydroxide from 2/98/1% v/v to 20/80/1% v/v) to afford Dasotraline (1) as an oil (0.23 g, 14% yield). Chromatographic purity (HPLC, area %): 85.3%.

### Example 6

### Preparation of (4S)-4-(3,4-dichlorophenyl)-N-[(1R)-1-(phenylethyl)-3,4-dihydronaphthalen-1(2H)-imine (Compound of Formula (3-B_{S}))

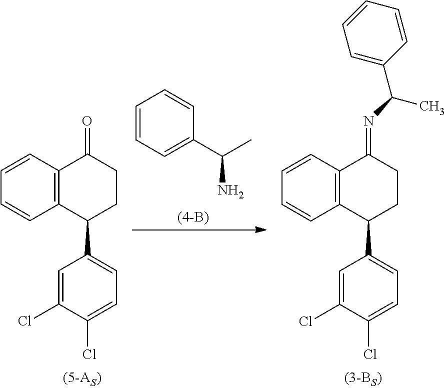

To a solution of the compound of Formula (5-A_{S}) (7.28 g, 25.0 mmol) in toluene (60 mL) was added the compound of Formula (4-B) (3.34 g, 27.0 mmol), and trifluoroacetic acid (0.25 mL). The resulting solution was refluxed with a Dean-Stark trap for 23 hours after which time ¹H NMR analysis of a sample of the solution showed 94% conversion. Additional trifluoroacetic acid (0.3 mL) was added and refluxing was continued for a further 7 hours, after which time additional compound of Formula (4-B) (0.4 g, 3.3 mmol) was added and refluxing continued for 16 hours. The reaction mixture was cooled to room temperature and concentrated to dryness at reduced pressure to provide the compound of Formula (3-B_{S}) that was used in Example 7 without further purification.

¹H-NMR of the compound of Formula (3-B_{S}) (300 MHz, CDCl₃) δ: 1.52 (3H, d, J=6.4 Hz), 2.00-2.10 (1H, m), 2.17-2.26 (1H, m), 2.41-2.50 (1H, m), 2.61-2.71 (1H, m), 4.10 (1H, t, J=5.2 Hz), 4.83 (1H, q, J=6.4 Hz), 6.85-6.91 (2H, m), 7.15-7.38 (7H, m), 7.44-7.47 (2H, m), 8.43 (1H, d, J=7.4 Hz).

### Example 7

### Preparation of (1R,4S)-4-(3,4-dichlorophenyl)-N-[(1R)-1-phenyl)ethyl]-1,2,3,4-tetrahydronaphthalen-1-amine (Compound of Formula (2-B_{S}))

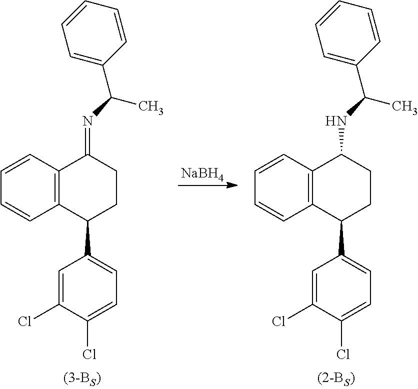 [SMILES: C[C@@H](/N=C1\CC[C@@H](c2ccc(Cl)c(Cl)c2)c2ccccc21)c1ccccc1 ; "NaBH4" ; C[C@@H](N[C@@H]1CC[C@@H](c2ccc(Cl)c(Cl)c2)c2ccccc21)c1ccccc1]

The compound of Formula (3-B_{S}) prepared in Example 6 was dissolved in a mixture of toluene (50 mL) and methanol (50 mL), cooled to −10° C., and sodium borohydride (1.48 g, 39.0 mmol) was added over about 2 hours while maintaining the reaction temperature below −3° C. The reaction mixture was then stirred for 19 hours at −9° C. Following this time, the reaction was quenched with hydrochloric acid (6 N, 6 mL) and adjusted to a pH of 1. The mixture was stirred for 50 minutes followed by slow addition of a saturated aqueous solution of sodium bicarbonate (120 mL) to adjust the pH to 8, and stirring continued for another 30 minutes. The phases were separated and the organic phase was concentrated to dryness to afford the compound of Formula (2-B_{S}) as an oil (10.23 g), which was purified by chromatography (gradient elution: ethyl acetate/hexane 5% v/v to 31% v/v) to afford the compound of Formula (2-B_{S}) as an oil (5.09 g, 51% yield, 2 steps). Chromatographic purity (HPLC, area %): 96.4%; chiral purity (chiral HPLC, area %) in favor of the (1R,4S)-isomer: 100%.

¹H-NMR of the compound of Formula (2-B_{S}) (300 MHz, CDCl₃) δ: 1.40 (3H, d, J=6.5 Hz), 1.51-1.58 (1H, m), 1.61-1.70 (1H, m), 1.80-1.88 (1H, m), 2.29-2.35 (1H, m), 3.85 (1H, t, J=4.9 Hz), 4.03-4.14 (2H, m), 6.77-6.82 (2H, m), 7.06-7.35 (7H, m), 7.41-7.43 (2H, m), 7.49 (1H, d, J=7.7 Hz).

### Example 8

### Preparation of (1R,4S)-4-(3,4-dichlorophenyl)-1,2,3,4-tetrahydronaphthalen-1-amine (Dasotraline (1))

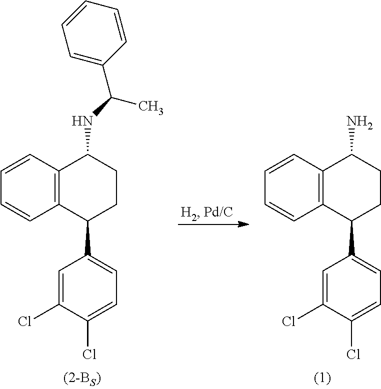 [SMILES: C[C@@H](N[C@@H]1CC[C@@H](c2ccc(Cl)c(Cl)c2)c2ccccc21)c1ccccc1 ; "H2, Pd/C" ; N[C@@H]1CC[C@@H](c2ccc(Cl)c(Cl)c2)c2ccccc21]

The compound of Formula (2-B_{S}) (1.41 g, 3.56 mmol), ethanol (10.5 mL), concentrated hydrochloric acid (0.51 g, 4.89 mmol) and palladium on carbon (10%, 0.05 g) were charged into a high pressure vessel equipped with a pressure gauge. After purging with hydrogen, the reaction mixture was stirred at room temperature under hydrogen at 50 psi for 5 hours. Following this time, the temperature was increased to 40° C. and the reaction continued for a further 81 hours at which point HPLC showed 87 area % of the desired product of Formula (1).

### Example 9

### Preparation of (4R,S)-4-(3,4-dichlorophenyl)-N-[(1R)-1-(4-methoxyphenyl)ethyl]-3,4-dihydronaphthalen-1(2H)-imine (compound of Formula (3-A_{RS}))

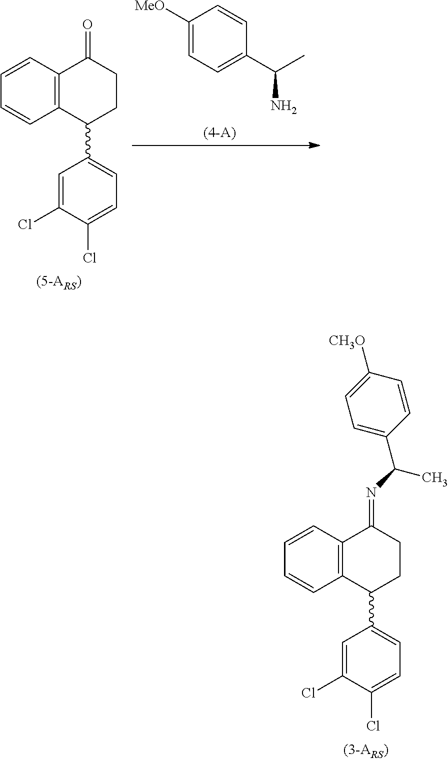

To a solution of the compound of Formula (5-A_{RS}) (racemic, 25.0 g, 85.9 mmol) in toluene (125 mL) was added the compound of Formula (4-A) (14.3 g, 94.4 mmol) and trifluoroacetic acid (0.5 mL). The resulting suspension was refluxed with a Dean-Stark trap for 28 hours after which time ¹H NMR analysis of a sample of the solution showed 97% conversion. The reaction mixture was cooled to room temperature and concentrated to dryness under reduced pressure to afford the compound of Formula (3-A_{RS}) as an oil (42.13 g, 86 wt % by assay, 100% yield). This material was used in Example 10 without further purification.

¹H-NMR of the compound of Formula (3-A_{RS}) (300 MHz, CDCl₃) δ: 1.49 (3H, d, J=6.2 Hz), 1.51 (3H, d, J=5.9 Hz), 1.99-2.12 (2H, m), 2.19-2.35 (2H, m), 2.42-2.62 (4H, m), 3.78 (3H, s), 3.79 (3H, s), 4.11-4.14 (2H, m), 4.79 (2H, q, J=6.5 Hz), 6.84-6.90 (8H, m), 7.15-7.38 (12H, m), 8.39-8.43 (2H, m).

### Example 10

### Preparation of (1R,4S)/(1R,4R)-4-(3,4-dichlorophenyl)-N-[(1S)-1-(4-methoxyphenyl)ethyl]-1,2,3,4-tetrahydronaphthalen-1-amine (compound of Formula (2-A_{RS}))

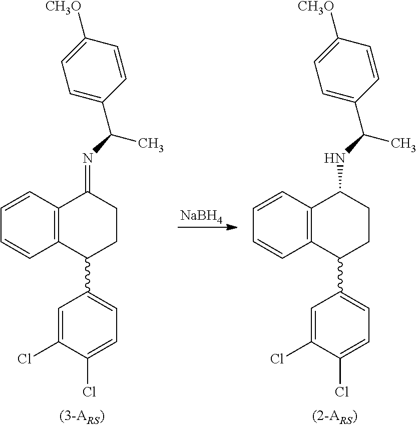

To a solution of the compound of Formula (3-A_{RS}) (42.0 g 86 wt % by assay, 85.1 mmol) prepared in Example 9 in toluene (110 ml) was added methanol (110 mL), and the solution was cooled to −13° C. Sodium borohydride (3.59 g, 94.9 mmol) was added over 4 hours while maintaining the temperature of the reaction mixture below −8° C. The reaction mixture was stirred for 1.5 hours at −8° C., and was subsequently quenched by the addition of concentrated hydrochloric acid (6 N, 20 mL) to adjust the pH to 1. The reaction mixture was allowed to warm to 8° C. and stirred for 30 minutes prior to slow addition of a saturated aqueous solution of sodium bicarbonate (100 mL), followed by an aqueous potassium carbonate solution (10% wt/v, 70 mL) to adjust the pH to ≥9. After stirring for an additional 25 minutes, the phases were separated, and the organic phase was concentrated to dryness to afford the compound of Formula (2-A_{RS}) as an oil (39.03 g, 90 wt % by assay, 97% yield). Chromatographic purity (HPLC, area %): 92.7 area %; chiral purity (chiral HPLC, area %): 46.3% (1R,4S), 48.3% (1R,4R), 3.03% (1S,4R), 2.4% (1S,4S)-isomer.

¹H-NMR compound of Formula (2-A_{S}) (300 MHz, CDCl₃) δ: 1.37 (3H, d, J=6.4 Hz), 1.48-1.56 (1H, m), 1.62-1.68 (1H, m), 1.80-1.88 (1H, m), 2.29-2.38 (1H, m), 3.79-3.85 (1H, m), 3.80 (3H, s), 4.00-4.06 (1H, m), 4.10-4.14 (1H, m), 6.79-6.82 (2H, m), 6.86-6.89 (2H, m), 7.06-7.14 (2H, m), 7.21-7.35 (4H, m), 7.49 (1H, d, J=7.7 Hz).

### Example 11

### Enrichment of (1R,4S)/(1R,4R)-4-(3,4-dichlorophenyl)-N-[(1S)-1-(4-methoxyphenyl)ethyl]-1,2,3,4-tetrahydronaphthalen-1-amine (compound of Formula (2-A_{RS})) in favour of the (1R,4S)-isomer

To a solution of the compound of Formula (2-A_{RS}) (5 g, 11.73 mmol) prepared in Example 10 in toluene (100 mL) was charged p-toluenesulfonic acid monohydrate (2.23 g, 11.73 mmol). A thick precipitate formed within minutes of the addition. The suspension was heated to about 70° C. at which point the suspension became thin. Heating was maintained at about 70° C. for two hours, slowly cooled to 50° C. over a period of 30 minutes, and then maintained for 4 hours at 50° C. The suspension was further cooled to room temperature over a period of 30 minutes and maintained at room temperature for 13 hours. The solid was collected by filtration, washed with toluene (2×10 mL), and dried in vacuo at about 40° C. for 20 hours to afford 4-(3,4-dichlorophenyl)-N-[(1S)-1-(4-methoxyphenyl)ethyl]-1,2,3,4-tetrahydronaphthalen-1-amine 4-toluenesulfonic acid salt (3.18 g, 44% yield) solid enriched in favour of the (1R,4R)-isomer. Chiral purity (chiral HPLC, area %): 1.9% (1R,4S), 91.1% (1R,4R), 3.0% (1S,4R), 3.9% (1S,4S)-isomer. Chiral purity of the mother liquor (chiral HPLC, area %): 84.8% (1R,4S), 10.9% (1R,4R), 3.0% (1S,4R), 1.4% (1S,4S)-isomer.

A 40 wt % portion (42.94 g) of the total mother liquor (106.52 g) comprising approximately 1.62 g (calculated) of the salt having the isomeric composition above was concentrated in vacuo at about 30° C. to a volume of 3.3 mL. To the concentrate was added 1,4-dioxane (24.5 mL) and the solution concentrated again in vacuo at about 30° C. to 13 mL. The clear orange solution (approximate molar ratio of 1,4-dioxane/toluene 97/3 by ¹H-NMR) was stirred at room temperature and the resulting thick paste was diluted with an additional portion of 1,4-dioxane (8.1 mL). The suspension was heated to 80° C. to form a solution, which was maintained at this temperature for 2 hours, slowly cooled to room temperature over a period of 2 hours, and then maintained at room temperature for 12 hours. The solid was collected by filtration, washed with 1,4-dioxane (2×3.3 mL), and dried in vacuo at about 40° C. for 23 hours to afford 4-(3,4-dichlorophenyl)-N-[(1S)-1-(4-methoxyphenyl)ethyl]-1,2,3,4-tetrahydronaphthalen-1-amine 4-toluenesulfonic acid salt (1.14 g) enriched in the (1R,4S)-isomer. Chiral purity (chiral HPLC, area %): 92.6% (1R,4S), 4.5% (1R,4R), 2.9% (1S,4R), 0% (1S,4S)-isomer.

The free base form of the 4-toluenesulfonic acid salt (1.14 g, 1.85 mmol) above was liberated by dissolving the salt in dichloromethane (20 mL) and washing with a saturated sodium bicarbonate solution (20 mL). The organic layer was then separated, dried over anhydrous sodium sulfate, and concentrated in vacuo at about 30° C. to afford the corresponding free form of Formula (2-A_{S}) as a clear, colourless oil (750 mg, 95% yield). This material was used directly in Example 12.

### Example 12

### Preparation of (1R,4S)-4-(3,4-dichlorophenyl)-1,2,3,4-tetrahydronaphthalen-1-amine (Dasotraline (1)) Hydrochloride Salt

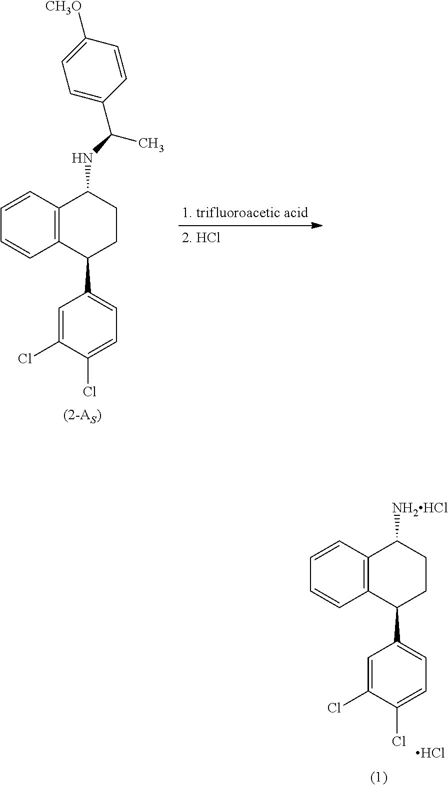

A solution of the compound of Formula (2-A_{S}) prepared in Example 11 (750 mg, 1.76 mmol) in trifluoroacetic acid (4.6 mL) was heated to reflux for 24 hours. The reaction mixture was cooled to room temperature, concentrated in vacuo at about 30° C., and the residue was diluted with dichloromethane (19 mL). A saturated aqueous solution of sodium bicarbonate (15 mL) was added and the mixture was stirred at room temperature for 2 hours. After phase separation, the organic layer was washed with aqueous saturated sodium bicarbonate solution (15 mL). The organic layer was then separated, dried over anhydrous sodium sulfate and filtered. Hydrogen chloride solution (4 M in 1,4-dioxane, 525 μL, 2.10 mmol) was added to the filtrate, causing formation of a purple suspension. The suspension was stirred at room temperature for 17 hours, and the solid was collected by filtration, washed with dichloromethane (2×1.5 mL), and dried in vacuo at about 40° C. for 5 hours to afford Dasotraline (1) hydrochloride as an off-white solid (410 mg, 71% yield). Chiral purity (chiral HPLC, area %): 95.7% (1R,4S), 0.9% (1R,4R), 3.4% (1S,4R), 0% (1S,4S)-isomer.

### Example 13

### Preparation of (1R,4S)/(1R,4R)-4-(3,4-dichlorophenyl)-1,2,3,4-tetrahydronaphthalen-1-amine (Compound of Formula (1_{RS}))

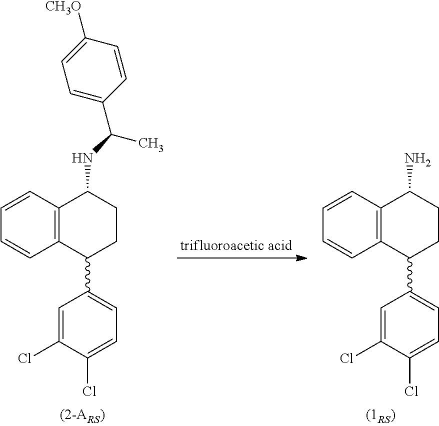

A portion of the compound of Formula (2-A_{RS}) (1.02 g, 2.39 mmol) prepared in Example 10 was dissolved in trifluoroacetic acid (6 mL) and heated at reflux for 4 days. The reaction mixture was cooled to room temperature, concentrated to dryness, and the residue was diluted with dichloromethane (50 mL). A saturated aqueous solution of sodium bicarbonate (21 mL) was added to adjust the pH to 8. After phase separation, the organic layer was dried over anhydrous sodium sulfate, filtered, and concentrated to dryness to afford the compound of Formula (1_{RS}) as an oil (0.87 g, 70% yield). Chromatographic purity (HPLC, area %): 90.4%; chiral purity (chiral HPLC, area %) 45.9% (1R,4S), 46.3% (1R,4R), 3.5% (1S,4R), 4.3% (1S,4S).

¹H-NMR of the compound of Formula (1_{RS}) (300 MHz, CDCl₃) δ: 1.57-1.85 (4H, m), 1.94-2.12 (2H, m), 2.26-2.35 (2H, m), 3.99-4.13 (4H, m), 6.79-6.97 (4H, m), 7.09-7.36 (8H, m), 7.44 (1H, d, J=7.7 Hz), 7.51 (1H, d, J=7.7 Hz).

### Example 14

### Resolution of Dasotraline (1) Isomers Using (1R)-(−)-10-Camphorsulfonic Acid

An isomeric mixture of the compound of Formula (1_{RS}) (400 mg, 1.44 mmol) prepared in accordance with the procedure in Example 13 and comprising 4.3% (1S,4S), 48.9% (1R,4R), 43.7% (1R,4S), and 3.2% (1S,4R) isomers (by chiral HPLC, area %), (1R)-(−)-10-camphorsulfonic acid (159 mg, 0.68 mmol), and ethyl acetate (1.7 mL) were combined to afford a white suspension. The suspension was heated to 40° C. for 1 hour, slowly cooled to room temperature over a period of 2 hours, and maintained at room temperature for 18 hours to afford a white suspension. The solid was collected by filtration, washed with ethyl acetate (1 mL), and dried in vacuo at 40° C. for 22 hours to afford Dasotraline (1R)-(−)-10-camphorsulfonate salt as a white solid (246 mg, 34% yield). Chiral purity (HPLC, area %): 95.6% (1R,4S), 0.7% (1S,4S), 3.5% (1R,4R), 0.2% (1S,4R).

### Example 15

### Resolution of Dasotraline (1) Isomers Using O,O′-Di-p-toluoyl-D-tartaric Acid

An isomeric mixture of 4-(3,4-dichlorophenyl)-1,2,3,4-tetrahydronaphthalen-1-amine (250 mg, 0.86 mmol) comprising 24% (1S,4S), 24% (1R,4R), 26% (1R,4S), and 26% (1S,4R) isomers (by chiral HPLC, area %), (+)-O,O″-Di-p-toluoyl-D-tartaric acid (83 mg, 0.21 mmol), and methyl tert-butyl ether (1 mL) were combined to afford a suspension. The suspension was heated to 40-45° C. for 1 hour and then cooled to room temperature over a period of 2 hours, and further cooled to 0-5° C. for 1 hour. Upon cooling to 0-5° C., the suspension thickened slightly. The solid was collected by filtration, washed with cold (0-5° C.) methyl tert-butyl ether (1 mL), and dried in vacuo at room temperature for 4 hours to afford Dasotraline (+)-O,O′-Di-p-toluoyl-D-tartrate salt as a white solid (5 mg, 0.007 mmol). Chiral purity (HPLC, area %) 59% (1R,4S), 22% (1S,4S), 17% (1R,4R), 0.9% (1S,4R).

What is claimed is:

1. A process for preparing Dasotraline (1): 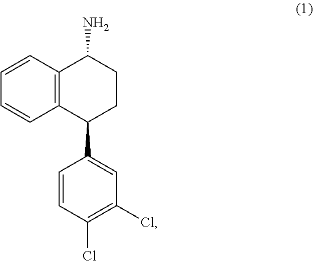 [SMILES: N[C@@H]1CC[C@@H](c2ccc(Cl)c(Cl)c2)c2ccccc21]
or a salt thereof, comprising deprotection of a compound of Formula (2): 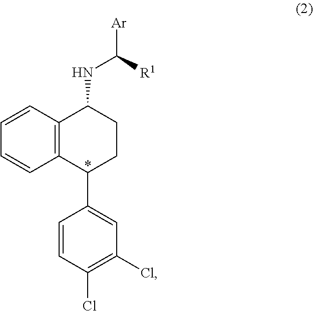
or a salt thereof,
wherein
the carbon atom marked with “*” is racemic, substantially racemic, or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and C₁-C₆ substituted alkyl.

2. The process of claim 1, wherein Ar is selected from the group consisting of phenyl, alkoxy-substituted phenyl, alkyl-substituted phenyl, and naphthyl.

3. The process of claim 2, wherein Ar is selected from the group consisting of 4-methoxyphenyl and phenyl.

4. The process of claim 3, wherein Ar is 4-methoxyphenyl.

5. The process of claim 4, wherein R¹ is methyl.

6. The process of claim 5, wherein the deprotection is conducted by acid-mediated hydrolysis comprising treatment of the compound of Formula (2) with an acid selected from the group consisting of trifluoroacetic acid, hydrochloric acid, and trifluoromethanesulfonic acid.

7. The process of claim 6, wherein the acid is trifluoroacetic acid.

8. The process of claim 1, wherein the carbon atom marked with “*” is enantiomerically enriched in the (S)-configuration and the compound of Formula (2) has a chiral purity in favour of the (1R,4S)-configuration of at least about 90%.

9. A process for preparing the compound of Formula (2): 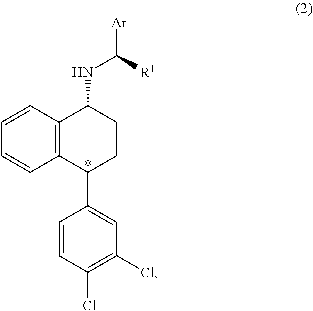
or a salt thereof, by reductive amination comprising reaction, in the presence of a solvent (S1), of a compound of Formula (5): 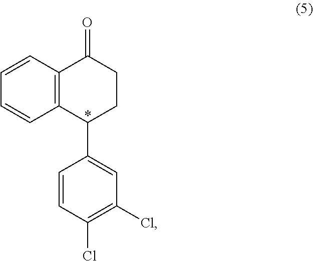 [SMILES: O=C1CCC(c2ccc(Cl)c(Cl)c2)c2ccccc21]
with an amine of Formula (4): 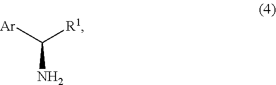
to afford a compound of Formula (3): 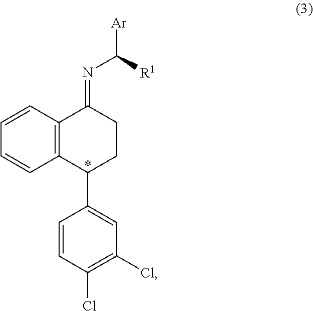
followed by reduction, in the presence of a solvent (S2), with a reductant to afford the compound of Formula (2), or a salt thereof,
wherein
the carbon atom marked with “*” is substantially racemic or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and C₁-C₆ substituted alkyl.

10. The process of claim 9, wherein Ar is selected from the group consisting of phenyl, alkoxy-substituted phenyl, alkyl-substituted phenyl, and naphthyl.

11. The process of claim 10, wherein Ar is selected from the group consisting of 4-methoxyphenyl and phenyl.

12. The process of claim 11, wherein Ar is 4-methoxyphenyl.

13. The process of claim 12, wherein R¹ is methyl.

14. The process of claim 9, wherein the reductant is selected from the group consisting of sodium borohydride, sodium cyanoborohydride, and sodium triacetoxyborohydride.

15. The process of claim 9, wherein the carbon atom marked with “*” is enantiomerically enriched in the (S)-configuration, and the compound of Formula (5) has a chiral purity in favor of the (4S)-configuration of at least 90%.

16. A compound of Formula (2): 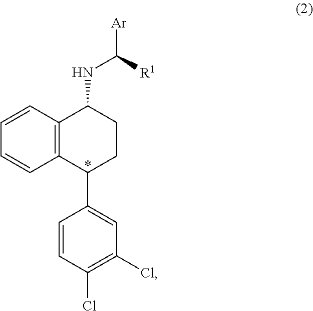
or a salt thereof,
wherein
the carbon atom marked with “*” is racemic, substantially racemic or enantiomerically enriched in the (S)-configuration;
Ar is selected from the group consisting of C₆-C₁₂ aryl and C₆-C₁₂ substituted aryl; and
R¹ is selected from the group consisting of C₁-C₆ alkyl and substituted C₁-C₆ alkyl.

17. The compound of claim 16, wherein Ar is selected from the group consisting of phenyl, alkoxy-substituted phenyl, alkyl-substituted phenyl, and naphthyl.

18. The compound of claim 17, wherein Ar is selected from the group consisting of 4-methoxyphenyl and phenyl.

19. The compound of claim 18, wherein Ar is 4-methoxyphenyl.

20. The compound of claim 19, wherein R¹ is methyl.
